(19) United States
(12) Patent Application Publication
(10) Pub. No.: US 2007/0213796 A1
(43) Pub. Date: Sep. 13, 2007

(54) Ring electrode

(76) Inventor: William J. McGinnis, Cincinnati, OH (US)

Correspondence Address: LADAS & PARRY LLP, 224 SOUTH MICHIGAN AVENUE, SUITE 1600, CHICAGO, IL, 60604, US

(21) Appl. No.: 11/372,928

(22) Filed: Mar. 10, 2006

(51) Int. Cl. A61N 1/00

(52) U.S. Cl. 607/117

(57) ABSTRACT

Devices for eliciting motor evoked potentials during anterior cervical discectomy and fusion procedures are provided, particularly an electrode for directly stimulating a vertebral post.

### TECHNICAL FIELD

This invention relates to surgical instruments for eliciting motor evoked potentials, and more particularly, to a surgical instrument for providing a stimulus directly to an anterior cervical vertebral body.

### TECHNICAL FIELD

This invention relates to surgical instruments for eliciting motor evoked potentials, and more particularly, to a surgical instrument for providing a stimulus directly to an anterior cervical vertebral body.

### BACKGROUND

Avoiding paralysis is a major intraoperative concern, and the most functionally significant impairments following spinal surgery are probably related to motor deficits. Motor evoked potentials (MEP) assess the motor systems from the motor cortex to the anterior horn cell and then by way of the peripheral nerves to the muscle, and monitoring these pathways should theoretically permit better detection of intraoperative motor function loss. Motor evoked potentials are recorded from either peripheral muscles (myogenic motor evoked potentials, MMEPs) or from peripheral nerves (neurogenic motor evoked potentials, NMEPs) and can be generated by applying either electrical or magnetic stimulation transcranially to the motor cortex or to the spinal cord.

Transcranial electrical stimulation of the motor cortex is a reliable method of eliciting motor evoked potentials, achieved by delivering brief high-voltage pulses through scalp electrodes. It is recorded from the spinal cord, the epidural space, the peripheral nerves and the musculature using conventional electromyographic and evoked potential averaging techniques. Transcranial magnetic stimulation is produced by placing a magnetic coil over the motor cortex. However, transcranial electrode placement and stimulation is time intensive and technically demanding intraoperatively. With transcranial magnetic or electrical stimulation, there is also concern that repetitive cortical stimulation can induce epileptic activity, neural damage and cognitive or memory dysfunction. Other situations of concern are patients with cardiac pacemakers and central venous or pulmonary artery catheterization. Furthermore, the clinical utility of transcranial motor evoked potentials is severely limited due to the large attenuation of evoked responses caused by most anesthetic agents.

Direct spinal cord stimulation has been used to assess the motor pathways with recordings obtained from the lower extremities using either surface or intramuscular electromyography to assess the motor pathways.

To date however, only posterior elements of the spinal cord such as such as the lamina have been stimulated, and it is believed that stimulating the posterior elements of the spinal cord may fail to achieve stimulation of the motor pathway, and thus may fail to produce an evoked potential.

Thus, there remains a need for a way monitoring the corticospinal tract's functional integrity intraoperatively.

It has been surprisingly found that a motor evoked potential is reliably elicited by providing a stimulus directly to the corticospinal tract (motor pathway), via stimulation of the anterior cervical vertebral bodies. The electrical resistance of the inherent positive charge of the bone is overcome and propagation of the electrical current follows the path of least resistance to the anterior surface of the spinal cord. The changes in electrical charge generated by the current, elicits conductive changes at the level of the corticospinal tract, thereby activating the motor pathways. The path of the stimulus when applied to the anterior cervical vertebral bodies is: vertebral body (anterior cervical spine)→anterior spinal cord→corticospinal tract (motor pathways).

It has also been surprisingly found that anterior cord stimulated motor evoked potentials are resistant to attenuation induced by general anesthesia. Furthermore, a device for providing stimulation directly to the spinal cord within a bony body or nerve tissue has so far eluded the industry.

A stimulating instrument and technique to reliably elicit motor evoked potentials intraoperatively would be of importance.

### SUMMARY OF THE INVENTION

The present invention features a stimulating electrode for reliably eliciting motor evoked potentials intraoperatively by stimulating a subject's corticospinal tract via an electrical stimulus delivered to the subject's vertebral body. A stimulating electrode is provided, comprising: a stimulating wire having a distal end connected to a ring portion adapted to be put in contact with a vertebral body post, and a proximal end capable of being put in contact with an electrical stimulus source. When the screw portion of the post is embedded in the subject's vertebral body, the stimulating wire is capable of providing an electric current to the subject's corticospinal tract via the vertebral body post.

Some implementations include one or more of the following features.

The ring portion of the electrode may have an inner and an outer diameter, and the stimulating wire extends to the inner diameter of the ring portion. The ring portion may also have an upper surface and a lower surface, wherein the upper surface of the ring portion comprises, or is covered by, an insulator, preferably rubber. The entire ring portion, save the lower surface of the ring portion may be covered with a rubber insulator or some other insulation material.

The ring portion may be made of platinum, stainless steel, amidester (Ha), litz, PVC, Teflon®, or nylon. In a preferred embodiment, the ring portion is made of gold.

The ring portion may be fitted around the screw portion.

The stimulating wire may be made of a threaded or non-threaded substance such as platinum, stainless steel, amidester (Ha), litz, PVC, Teflon®, nylon, gold, silver/silver chloride, silver, titanium, tin and copper. The stimulating wire may be coated with insulation material.

The stimulating wire may comprise a male connector and a female connector interposed between the proximal end and the distal end, wherein when the female connector receives insertion of the male connector, a stimulus is provided to the ring electrode. The female connector may be distal to the ring portion.

The ring portion may have an attached handle. The ring portion may be connected to the handle via a bendable joint. The handle and the bendable joint may be covered with an insulating material such as rubber.

Other features and advantages of the invention will be apparent from the detailed description and drawings, and from the claims.

### DESCRIPTION OF DRAWINGS

FIG. 1 illustrates one embodiment of a stimulating ring electrode for stimulating a vertebral post.

FIG. 2 shows one view of the stimulus ring electrode fitted around a vertebral post screw.

FIG. 3 shows a view of the stimulus ring electrode fitted around a vertebral post screw embedded in a vertebral body.

Like reference symbols in the various drawings indicate like elements.

### DETAILED DESCRIPTION

The invention provides an electrode for directly stimulating osseus structures, providing a stimulus to the anterior cervical spine via the anterior vertebral bodies and reliably eliciting motor evoked potentials intraoperatively.

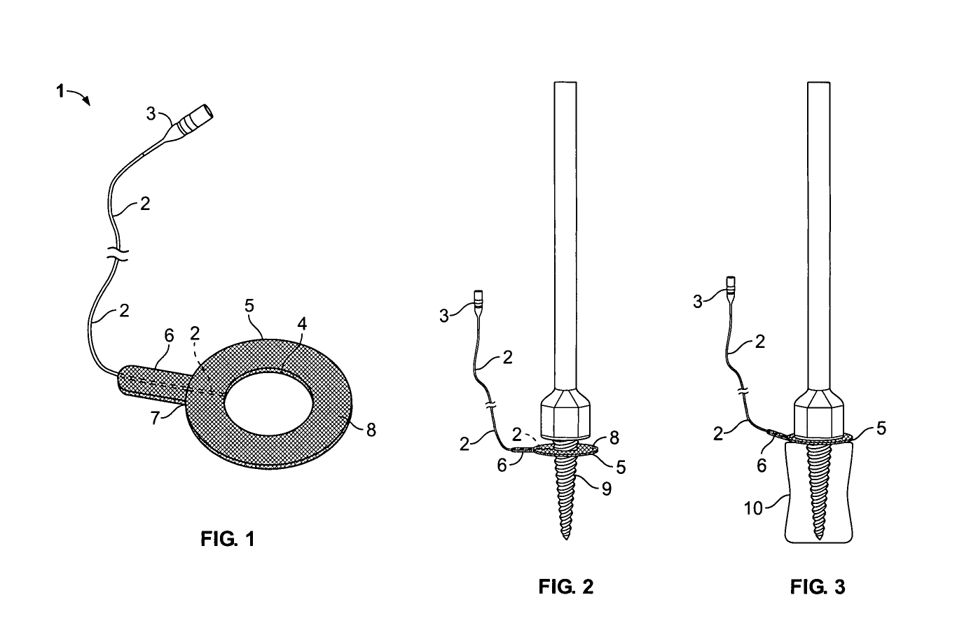

FIG. 1 shows one embodiment of the stimulating ring electrode (1) comprising stimulation wire (2) connected proximally via DIN connector (3) to an electrical stimulator source, and connected distally to inner diameter (4) of ring portion (5) attached to handle (6). Between (5) and (6) is bendable joint (7) As FIG. 1 shows, wire (2) extends through the inside of handle (6) and ring (5) to reach inner diameter (4). When ring (5) is made to be in contact with a vertebral post screw (see (9) in FIG. 2) embedded in an osseous or tissue material, an electrical stimulus is delivered to the proximal osseous or tissue surface via the vertebral post. Shown in this embodiment, ring (4) has upper surface (8) comprising a rubber insulator, but might also be covered with a Teflon® or plastic coating or similar non-conducting material. Inner diameter (4) of ring (5) is approximately 3 mm. The outer diameter of ring (5) is approximately 5 mm. The handle is approximately 3 mm in length.

In the view shown in FIG. 2, a stimulus ring electrode as shown in FIG. 1 (1) is fitted around vertebral body screw (9). In FIG. 3, the stimulus ring electrode is fitted around a vertebral body screw that is embedded in vertebral body (10). It should be understood therefore that the stimulus, in being delivered via the vertebral post to the anterior cervical vertebral body exposed during the surgical procedure, thereby delivers an electrical stimulus to the corticospinal tract.

The preponderance of anterior discectomies and fusions at level specific sites employ the use of devices such as a vertebral body spreader, or vertebral body post distractor (vertebral body post), to expose the disc space. Other commonly used devices during spinal surgeries are pins, posts and needles that are capable of being embedded in a subject's osseus structure such as a vertebral body. The inventive ring electrode is contemplated as being adapted to fit around such commonly used devices, wherein an electrical stimulus is provided to the anterior cervical spine, via the ring portion fitted around the screw portion of such surgical instruments when the screw portion is screwed down into the subject's vertebral body.

In a preferred embodiment therefore, the electrode comprises a stimulating wire connected distally to a ring portion adapted for being put in contact with the screw portion of a vertebral distractor or post, and proximally capable of being connected to an electrical stimulator source, wherein when the ring portion is in contact with the screw portion of a vertebral distractor or post. In a highly preferred embodiment, the ring portion is fitted around the screw portion of a vertebral post.

The electrode thus enables muscle or myogenic motor evoked potentials from the upper thenar musculature and lower tibialis anterior musculature to be recorded.

With such an electrode, delivering an electrical stimulation to a vertebral body using a vertebral body post as a medium, current exceeds the impedance of the bone and overcomes the impedance, travels out of the bone, excites the spinal cord which is recorded in the peripheral musculature over muscle using needle electrodes in the upper extremities and lower extremities.

As shown in FIG. 3, when the vertebral post is embedded in a subject's osseous or tissue structure the stimulating wire is capable of providing a current to the proximal osseous or tissue surface via the ring portion when the ring portion is fitted onto the screw portion of a vertebral post. Thus, the stimulus is delivered to the anterior cervical vertebral body exposed during the surgical procedure.

In a preferred embodiment, the ring portion has an inner and an outer diameter, and the stimulating wire extends to the inner diameter of the ring portion. In a highly preferred embodiment, the stimulating wire passes through the middle of the ring portion, extending to the inner diameter of the ring portion.

In yet another embodiment, the ring portion comprises an upper surface and a lower surface, the upper surface of the ring comprising an insulator, preferably rubber. In a further embodiment, the ring portion is covered with an insulating material, preferably rubber, excepting only the lower surface of the ring portion.

In a preferred embodiment, the ring portion is made of titanium, platinum, stainless steel, silver, silver/silver chloride, tin, copper or gold. In a highly preferred embodiment, the ring is made of gold.

In another highly preferred embodiment, the ring portion is fitted around the screw portion of the vertebral post. In a further preferred embodiment, the ring portion is connected to a handle, preferably the handle is covered with an insulating material. In an alternative embodiment, the ring portion is connected to the handle via a bendable joint. In yet a further embodiment, the stimulating wire passes through the middle of the handle, and through the middle of the ring portion, extending to the inner diameter of the ring portion.

Alternatively, the stimulating wire may further comprise a male connector and a female connector interposed between the proximal end and the distal end, wherein when the female connector receives insertion of the male connector, the current is provided to the ring electrode. In one embodiment, the female connector is distal to the ring portion.

The stimulating wire is preferably made of a wire such as amidester (Ha), litz, nylon/FEP (fluorinated ethylene propylene) or nylon/nylon, but may also be made of any suitable means for conducting current from the stimulator source to its terminal end such as thread conductive materials, gold, silver/silver chloride, silver, titanium, tin and copper. In another preferred embodiment, the wire is coated with insulation material.

While the instant invention is drawn to embodiments of a ring electrode for delivering a current to an osseus structure such as a vertebral element, the invention should not be limited by the above described embodiments. For instance, the electrode could effectively be clipped to the post.

What is claimed is:

1. A stimulating electrode for delivering an electrical stimulus to a subject's corticospinal tract, comprising:
a stimulating wire having a distal end connected to a ring portion adapted to be put in contact with a vertebral body post, and a proximal end capable of being put in contact with an electrical stimulus source.

2. The electrode of claim 1, wherein the ring portion is adapted for fitting around the screw portion of the vertebral body post.

3. The stimulating electrode of claim 2, wherein the ring portion has an inner and an outer diameter, and the stimulating wire extends to the inner diameter of the ring portion.

4. The stimulating electrode of claim 3, wherein the ring portion has an upper surface and a lower surface, the upper surface further comprising a rubber insulator.

5. The stimulating electrode of claim 1, wherein the wire is made of a substance selected from the group consisting of platinum, stainless steel, amidester (Ha), litz, PVC, Teflon®, nylon, gold, silver/silver chloride, silver, titanium, tin and copper.

6. The electrode of claim 1, wherein the wire is coated with an electrical insulation material.

7. The electrode of claim 1, wherein the ring portion is made of a substance selected from the group consisting of titanium, platinum, stainless steel, silver, silver/silver chloride, tin, copper and gold.

8. The electrode of claim 6, wherein the substance is gold.

9. The electrode of claim 2, wherein the stimulating wire further comprises a male connector and a female connector interposed between the proximal end and the distal end, wherein when the female connector receives the male connector, and the current is provided to the ring electrode.

10. The electrode of claim 9, wherein the female connector is distal to the ring portion.

12. The electrode of claim 2, further comprising a handle connected to the ring portion.

13. The electrode of claim 12, wherein the ring portion is connected to the handle via a bendable joint.
